(19) United States
(12) Patent Application Publication
(10) Pub. No.: US 2025/0325610 A1
(43) Pub. Date: Oct. 23, 2025

(54) Compositions Containing Cannabis and Hericium Extracts for the Improvement of Mental Health and Treatment of CNS Disorders

(71) Applicant: DrinkBrez, LLC, West Palm Beach, FL (US)

(72) Inventors: James Preston Campbell, Goodlettsville, TN (US); Travis Ryan Duncan, Sarasota, FL (US); Aaron James Nosbisch, West Palm Beach, FL (US)

(73) Assignee: DrinkBrez, LLC, West Palm Beach, FL (US)

(21) Appl. No.: 19/184,427

(22) Filed: Apr. 21, 2025

Related U.S. Application Data
(60) Provisional application No. 63/636,763, filed on Apr. 20, 2024.

(51) Int. Cl. A61K 36/07; A61K 31/00; A61K 36/185; A61K 45/06; A61P 25/22; A61P 25/32

(52) U.S. Cl. CPC A61K 36/07; A61K 31/658; A61K 36/3482; A61K 45/06; A61P 25/22; A61P 25/32

(57) ABSTRACT

The present disclosure is directed to a beverage composition comprising from a cannabis extract and a Hericium erinaceus extract. The present disclosure also relates to methods of reducing a withdrawal symptom and/or reducing an addictive behavior comprising administering a therapeutically effective amount of the aforementioned beverage composition to a subject having the withdrawal symptom and/or the addictive behavior, methods for treating alcoholism in a subject comprising administering a therapeutically effective amount of the aforementioned beverage composition to the subject, and method of producing altered perception and/or reduced stress while ameliorating a side effect of THC overconsumption in a subject comprising administering a therapeutically effective amount of the aforementioned beverage composition to the subject.

### CROSS-REFERENCE TO RELATED APPLICATIONS

This application claims the benefit of U.S. Provisional Application No. 63/636,763, filed on Apr. 20, 2024, which is incorporated herein, in its entirety, by reference.

### FIELD

This disclosure relates to the field of therapeutic beverages and other orally administered treatments.

### BACKGROUND

Substance Use Disorder (SUD) is a chronic and relapsing disease characterized by compulsive substance use, loss of control over substance intake, and negative emotional states, including anxiety, in the absence of a substance, such as alcohol or opiates. Current treatment approaches for SUD often focus on behavioral therapies and medications with limited efficacy and potential side effects (Butelman & Kreek, Expert Opin. Emerg. Drugs., 22:301-15 (2017)).

There is a growing need for novel therapeutic approaches to address the complex neurobiological mechanisms underlying different types of SUD including opiate use disorder (OUD) and alcohol use disorder (AUD). The anxiolytic properties of cannabis, particularly those of cannabidiol (CBD) and delta-9 Tetrahydracannabinol (THC) when part of a full-spectrum extract, offer a promising avenue for reducing withdrawal symptoms, a primary contributor to relapse (De Ternay et al., Front. Pharmacol., 10:627 (2019)). Additionally, bioactive compounds derived from the medicinal mushroom Hericium erinaceus (Lion's Mane), possess the ability to activate GABA signaling and modulate kappa opioid receptors (KOR), which play a pivotal role in addiction pathways (Banks, Handb. Exp. Pharmacol., 258:147-65 (2020); Volkow et al., Physiol. Rev. 99:2115-40 (2019)). Therefore, targeting KOR with active compounds in Hericium extracts could mitigate the withdrawal and addiction associated SUD and increase patient compliance through synergistic effects of Hericium with cannabis on GABA signaling and resultant anxiolytic effects.

### SUMMARY

One aspect is for a beverage composition comprising: (a) from about 5 mg to about 100 mg of a cannabis extract; and (b) from about 100 mg to about 3,000 mg of a Hericium erinaceus extract; per about 222 mL to about 355 mL of beverage composition volume.

In some embodiments, the cannabis extract comprises tetrahydrocannabinol (THC) and cannabidiol (CBD); and in some embodiments, the THC and the CBD are present in the cannabis extract in a ratio in a range of about 1:50 to about 1:1. In some embodiments, the H. erinaceus extract comprises: (i) one or more erinacines; (ii) one or more hericenones; or (iii) a combination thereof; in some embodiments, the one or more erinacines are erinancine A, erinancine B, erinancine C, erinancine D, erinancine E, erinancine F, erinancine G, erinancine H, erinancine I, erinancine J, erinancine K, erinancine P, erinancine Q, erinancine S, erinancine U, or a combination thereof; and in some embodiments, the one or more hericenones are hericenone A, hericenone B, hericenone C, hericenone D, hericenone E, hericenone F, hericenone G, hericenone H, hericenone I, hericenone J, hericenone K, or a combination thereof.

Another aspect is for a method of reducing a withdrawal symptom and/or reducing an addictive behavior comprising administering a therapeutically effective amount of the aforementioned beverage composition to a subject having the withdrawal symptom and/or the addictive behavior.

A further aspect is for a method for treating alcoholism in a subject comprising administering a therapeutically effective amount of the aforementioned beverage composition to the subject. In some embodiments, the beverage composition replaces alcohol consumption by the subject.

An additional aspect is for a method of producing altered perception and/or reduced stress while ameliorating a side effect of THC overconsumption in a subject comprising administering a therapeutically effective amount of the aforementioned beverage composition to the subject.

Other objects and advantages will become apparent to those skilled in the art upon reference to the detailed description that hereinafter follows.

### BRIEF DESCRIPTION OF THE DRAWINGS

FIG. 1 shows Theta Power of Lion's Mane alone (15 and 30 minutes), THC alone (15 and 30 minutes), and THC+Lion's Mane (15 and 30 minutes).

FIG. 2 shows bar charts of demographic features of participants. FIG. 2A—sex; FIG. 2B—parent/guardian of a child; FIG. 2C—number of children in household; FIG. 2D ethnicity; FIG. 2E—education; FIG. 2F—martial status; FIG. 2G—occupation; FIG. 2H—income.

FIG. 3 shows the changes over time in scores over the course of time from before product use to during product use. Note that the dotted lines represent a random sample of 30 participants and the solid line represents the average changes in scores for all participants over time.

FIG. 4 shows the changes over time in scores over the course of time from before product use to during product use. Note that the dotted lines represent a random sample of 30 participants and the solid line represents the average changes in scores for all participants over time.

FIG. 5 shows the changes over time in scores over the course of time from before product use to during product use. Note that the dotted lines represent a random sample of 30 participants and the solid line represents the average changes in scores for all participants over time.

FIG. 6 shows the changes over time in scores over the course of time from before product use to during product use. Note that the dotted lines represent a random sample of 30 participants and the solid line represents the average changes in scores for all participants over time.

FIG. 7 shows a visual display of Alcohol Consumption predicted probabilities per week.

FIG. 8 shows the estimated average scores (points) and 95% confidence intervals (error bars) for the “No Product Use” (left) and “Product Use” (right) conditions, as predicted by the linear mixed-effects model.

FIG. 9 shows the estimated average scores (points) and 95% confidence intervals (error bars) for the “No Product Use” (left) and “Product Use” (right) conditions, as predicted by the linear mixed-effects model.

FIG. 10 shows the estimated average scores (points) and 95% confidence intervals (error bars) for the “No Product Use” (left) and “Product Use” (right) conditions, as predicted by the linear mixed-effects model.

### DETAILED DESCRIPTION

Applicant has solved the stated problem. Specifically, the present disclosure provides a beverage formulated with a unique combination of cannabis extracts and Hericium extract for affecting cognitive function including the treatment of substance use disorder (SUD) and other neurological disorders such as depression and general anxiety. The formulation leverages the anxiolytic effects of cannabis to alleviate withdrawal symptoms which are enhanced by Hericium extracts through synergistic anxiolytic effects via gamma-aminobutryic acid (GABA) signaling or though modulation of kappa opioid receptors (KOR) to reduce addictive behaviors. Furthermore, a beverage form factor facilitates convenient administration that improves patient compliance and provides a direct substitution for alcoholic drinks.

### Definitions

Applicant specifically incorporates the entire contents of all cited references in this disclosure. Further, when an amount, concentration, or other value or parameter is given as either a range or a list of upper values and lower values, this is to be understood as specifically disclosing all ranges formed from any pair of any upper range limit or value and any lower range limit or value, regardless of whether ranges are separately disclosed. Where a range of numerical values is recited herein, unless otherwise stated, the range is intended to include the endpoints thereof, and all integers and fractions within the range. It is not intended that the scope of the present disclosure be limited to the specific values recited when defining a range.

The indefinite articles “a” and “an”, as used herein in the specification and in the claims, unless clearly indicated to the contrary, should be understood to mean “at least one”.

The phrase “and/or”, as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B”, when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of”, or, when used in the claims, “consisting of”, will refer to the inclusion of exactly one element of a number or list of elements. In general, the term “or” as used herein shall only be interpreted as indicating exclusive alternatives (i.e., “one or the other but not both”) when preceded by terms of exclusivity, “either”, “one of”, “only one of”, “exactly one of”. “Consisting essentially of”, when used in the claims, shall have its ordinary meaning as used in the field of patent law.

The term “about” as used herein when referring to a measurable value such as an amount, a temporal duration, and the like, is meant to encompass variations of ±20%, ±10%, ±5%, ±1%, or ±0.1% from the specified value, as such variations are appropriate to perform the disclosed methods.

The term “symptom,” as used herein, refers to any morbid phenomenon or departure from the normal in structure, function, or sensation, experienced by the patient and indicative of disease.

As used herein, the term “treating” may include prophylaxis of the specific disease, disorder, or condition, or alleviation of the symptoms associated with a specific disease, disorder or condition and/or preventing or eliminating the symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein. The term “treating” includes therapeutic treatments. A treatment can be therapeutic if it is intended to diminish, ameliorate, or stabilize the existing unwanted condition or side effects thereof.

The terms “addiction” and “dependence” are used interchangeably to refer to the patient's inability to stop using the opioid or opioid-like drug, even when it would be in his/her best interest to stop. The Diagnostic and Statistical Manual of Mental Disorders Text Revision (DSMIV-TR) criteria for dependency include: Dependence or significant impairment or distress, as manifested by 3 or more of the following during a 12 month period: 1. Tolerance or markedly increased amounts of the substance to achieve intoxication or desired effect or markedly diminished effect with continued use of the same amount of substance. 2. Withdrawal symptoms or the use of certain substances to avoid withdrawal symptoms. 3. Use of a substance in larger amounts or over a longer period than was intended. 4. Persistent desire or unsuccessful efforts to cut down or control substance use. 5. Involvement in chronic behavior to obtain the substance, use the substance, or recover from its effects. 6. Reduction or abandonment of social, occupational or recreational activities because of substance use. 7. Use of substances even though there is a persistent or recurrent physical or psychological problem that is likely to have been caused or exacerbated by the substance.

“Drug withdrawal” refers to a group of symptoms that occur upon the abrupt discontinuation or sudden decrease in intake of medications or recreational drugs. Consequently, “opioid withdrawal” refers to the group of symptoms that occur upon the dramatic reduction, abrupt discontinuation or decrease in intake of opioids or opiates. Withdrawal symptoms may also start between doses. Withdrawal symptoms from opioids include but are not limited to anxiety, depression, sweating, vomiting, and diarrhea, muscle cramping, agitation, insomnia, yawning dilated pupils, goose bumps, abdominal cramping, runny nose and increased tearing, for example.

By “altered perception” is meant altered sensory experiences, frequently with heightened awareness of sensory input but with diminished control over what is being experienced. Altered perception includes how an individual experiences time, space, and one's senses.

### Beverage Composition

In some embodiments, the beverage composition comprises from about 5 mg to about 100 mg of a cannabis extract; and from about 100 mg to about 3,000 mg of a H. erinaceus (also known as lion's mane, yamabushitake, bearded tooth fungus, or bearded hedgehog) extract; per about 222 mL to about 355 mL of beverage composition volume.

Cannabinoids can be isolated by extraction from cannabis plants. Plants in the cannabis genus include Cannabis sativa, Cannabis ruderalis, and Cannabis indica. These plants are the natural sources of cannabinoids. Cannabinoids are also available in synthetic forms.

Cannabis oil can be obtained by cold pressing and/or extracting the entire Cannabis sativa L. plant or hemp plant. Depending on the specific cultivar, the resulting cannabis oil may contain certain percentages of THC and CBD, as well as other cannabinoids.

A cannabis oil can be produced by solvent extraction, e.g. ultrasonic extraction or ultrasonically-assisted supercritical CO₂ extraction of cannabis or hemp. For the production of hash oil, preferably dry, decarboxylated plant material is used. The following liquids are frequently used solvents for hash oil extraction: ethanol, chloroform, dichloromethane, petroleum ether, naphtha, benzene, butane, methanol, isopropanol, and olive oil. There are at least 113 identified types of cannabinoids in cannabis. Cannabinoids are active phytochemical substances with psychoactive and/or medical effects. THC or tetrahydrocannabinol is the primary psychoactive compound in cannabis. CBD or cannabidiol is another important ingredient with therapeutic effects. Cannabinoids are lipophilic and potentially acid-labile compounds. Cannabinoids are insoluble in water, but they are very soluble in non-polar solvents (e.g. fat and oil).

After a short-path distillation process, a subsequent manufacturing process can involve the isolation of CBD as a crystalline powder. The manufacturing process for CBD isolate can involve dissolving the full spectrum distillate in a solvent, then slowing modifying the solvent level and temperature to cause the highly concentrated levels of CBD to precipitate from solution as a combination of crystallized CBD and an amorphous CBD rich powder. This CBD isolate is of a very high purity, for example having a greater than 99.5 wt % pure CBD. The other cannabinoids from the full spectrum distillate remain in the Isolate solvent in an intermediate product known as Mother Liquor.

In some embodiments, the beverage composition can comprise the cannabis extract in an amount of about 5 mg, about 6 mg, about 7 mg, about 8 mg, about 9 mg, about 10 mg, 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 26 mg, about 27 mg, about 28 mg, about 29 mg, about 30 mg, about 31 mg, about 32 mg, about 33 mg, about 34 mg, about 35 mg, about 36 mg, about 37 mg, about 38 mg, about 39 mg, about 40 mg, about 41 mg, about 42 mg, about 43 mg, about 44 mg, about 45 mg, about 46 mg, about 47 mg, about 48 mg, about 49 mg, about 50 mg, about 51 mg, about 52 mg, about 53 mg, about 54 mg, about 55 mg, about 56, about 57 mg, about 58 mg, about 59 mg, about 60 mg, about 61 mg, about 62 mg, about 63 mg, about 64 mg, about 65 mg, about 66 mg, about 67 mg, about 68 mg, about 69 mg, about 70 mg, about 71 mg, about 72 mg, about 73 mg, about 74 mg, about 75 mg, about 76 mg, about 77 mg, about 78 mg, about 79 mg, about 80 mg, about 81 mg, about 82 mg, about 83 mg, about 84 mg, about 85 mg, about 86 mg, about 87 mg, about 88 mg, about 89 mg, about 90 mg, about 91 mg, about 92 mg, about 93 mg, about 94 mg, about 95 mg, about 96 mg, about 97 mg, about 98 mg, about 99 mg, or about 100 mg.

Cannabis extract content values can also be expressed as ranges, e.g., about 5 mg to about 100 mg, about 10 mg to about 100 mg, about 15 mg to about 100 mg, about 20 mg to about 100 mg, about 25 mg to about 100 mg, about 30 mg to about 100 mg, about 35 mg to about 100 mg, about 40 mg to about 100 mg, about 45 mg to about 100 mg, about 50 mg to about 100 mg, about 55 mg to about 100 mg, about 60 mg to about 100 mg, about 65 mg to about 100 mg, about 70 mg to about 100 mg, about 75 mg to about 100 mg, about 80 mg to about 100 mg, about 85 mg to about 100 mg, about 90 mg to about 100 mg, about 91 mg to about 100 mg, about 92 mg to about 100 mg, about 93 mg to about 100 mg, about 94 mg to about 100 mg, about 95 mg to about 100 mg, about 96 mg to about 100 mg, about 97 mg to about 100 mg, about 98 mg to about 100 mg, about 99 mg to about 100 mg, about 5 mg to about 95 mg, about 5 mg to about 90 mg, about 5 mg to about 85 mg, about 5 mg to about 80 mg, about 5 mg to about 75 mg, about 5 mg to about 70 mg, about 5 mg to about 65 mg, about 5 mg to about 60 mg, about 5 mg to about 55 mg, about 5 mg to about 50 mg, about 5 mg to about 45 mg, about 5 mg to about 40 mg, about 5 mg to about 35 mg, about 5 mg to about 30 mg, about 5 mg to about 25 mg, about 5 mg to about 20 mg, about 5 mg to about 15 mg, about 5 mg to about 14 mg, about 5 mg to about 13 mg, about 5 mg to about 12 mg, about 5 mg to about 11 mg, about 5 mg to about 10 mg, about 5 mg to about 9 mg, about 5 mg to about 8 mg, about 5 mg to about 7 mg, or about 5 mg to about 6 mg.

Lion's mane mushroom (H. erinaceus) is an edible mushroom that is well-established as a culinary and medicinal mushroom for brain and nerve health. Lion's mane mushrooms, and its powders, extracts, tinctures, etc. are readily available from numerous third parties and commercial sources. Lion's mane mushroom compositions and methods for making and using Lion's mane mushroom extracts are well documented in the literature. See, e.g., International Publication Number WO 2011/094579; Japanese Patent JP4965801; Japanese Patent JP2006117541; Japanese Patent JP2003212790; Lai et al., Neurotrophic properties of the Lion's mane medicinal mushroom, Hericium erinaceus (Higher Basidiomycetes) from Malaysia (2013) Int J Med Mushrooms 15 (6): 539-5; Kushairi et al. Lion's Mane Mushroom, Hericium erinaceus (Bull.: Fr.) Pers. Suppresses H₂O₂-Induced Oxidative Damage and LPS-Induced Inflammation in HT22 Hippocampal Neurons and BV2 Microglia (August 2019) Antioxidants (Basel) 1;8 (8): 261; Mendel Friedman, Chemistry, Nutrition, and Health-Promoting Properties of Hericium erinaceus (Lion's Mane) Mushroom Fruiting Bodies and Mycelia and Their Bioactive Compounds (2015) J. Agric. Food Chem. 63, 32, 7108-7123; and, Syed Asim Shah Bacha et al., Lion's mane mushroom; new addition to food and natural bounty for human wellness: A review (October 2018) International Journal of Biosciences Vol. 13, No. 4:396-402. In some embodiments, Lion's mane fruiting bodies are compressed, ultrasonically extracted, and nanoemulsified into a concentrated nanoemulsion to produce extracts usefule in the present beverage compositions.

In any of the foregoing embodiments, the beverage composition can include H. erinaceus extract at about 100 mg, about 110 mg, about 120 mg, about 130 mg, about 140 mg, about 150 mg, about 160 mg, about 170 mg, about 180 mg, about 190 mg, about 200 mg, about 300 mg, about 400 mg, about 500 mg, about 600 mg, about 700 mg, about 800 mg, about 900 mg, about 1,000 mg, about 1,100 mg, about 1,200 mg, about 1,300 mg, about 1,400 mg, about 1,500 mg, about 1,600 mg, about 1,700 mg, about 1,800 mg, about 1,900 mg, about 2,000 mg, about 2,100 mg, about 2,200 mg, about 2,300 mg, about 2,400 mg, about 2,500 mg, about 2,600 mg, about 2,700 mg, about 2,800 mg, about 2,900 mg, about 2,910 mg, about 2,920 mg, about 2,930 mg, about 2,940 mg, about 2,950 mg, about 2,960 mg, about 2,970 mg, about 2,980 mg, about 2,990 mg, or about 3,000 mg.

H. erinaceus extract content values can also be expressed as ranges, e.g., about 100 mg to about 3,000 mg, about 100 mg to about 2,900 mg, about 100 mg to about 2,800 mg, about 100 mg to about 2,700 mg, about 100 mg to about 2,600 mg, about 100 mg to about 2,500 mg, about 100 mg to about 2,400 mg, about 100 mg to about 2,300 mg, about 100 mg to about 2,200 mg, about 100 mg to about 2,100 mg, about 100 mg to about 2,000 mg, about 100 mg to about 1,900 mg, about 100 mg to about 1,800 mg, about 100 mg to about 1,700 mg, about 100 mg to about 1,600 mg, about 100 mg to about 1,500 mg, about 100 mg to about 1,400 mg, about 100 mg to about 1,300 mg, about 100 mg to about 1,200 mg, about 100 mg to about 1,100 mg, about 100 mg to about 1,000 mg, about 100 mg to about 900 mg, about 100 mg to about 800 mg, about 100 mg to about 700 mg, about 100 mg to about 600 mg, about 100 mg to about 500 mg, about 100 mg to about 400 mg, about 100 mg to about 300 mg, about 100 mg to about 200 mg, about 100 mg to about 190 mg, about 100 mg to about 180 mg, about 100 mg to about 170 mg, about 100 mg to about 160 mg, about 100 mg to about 150 mg, about 100 mg to about 140 mg, about 100 mg to about 130 mg, about 100 mg to about 120 mg, about 100 mg to about 110 mg, about 200 mg to about 3,000 mg, about 300 mg to about 3,000 mg, about 400 mg to about 3,000 mg, about 500 mg to about 3,000 mg, about 600 mg to about 3,000 mg, about 700 mg to about 3,000 mg, about 800 mg to about 3,000 mg, about 900 mg to about 3,000 mg, about 1,000 mg to about 3,000 mg, about 1,100 mg to about 3,000 mg, about 1,200 mg to about 3,000 mg, about 1,300 mg to about 3,000 mg, about 1,400 mg to about 3,000 mg, about 1,500 mg to about 3,000 mg, about 1,600 mg to about 3,000 mg, about 1,700 mg to about 3,000 mg, about 1,800 mg to about 3,000 mg, about 1,900 mg to about 3,000 mg, about 2,000 mg to about 3,000 mg, about 2,100 mg to about 3,000 mg, about 2,200 mg to about 3,000 mg, about 2,300 mg to about 3,000 mg, about 2,400 mg to about 3,000 mg, about 2,500 mg to about 3,000 mg, about 2,600 mg to about 3,000 mg, about 2,700 mg to about 3,000 mg, about 2,800 mg to about 3,000 mg, about 2,900 mg to about 3,000 mg, about 2,910 mg to about 3,000 mg, about 2,920 mg to about 3,000 mg, about 2,930 mg to about 3,000 mg, about 2,940 mg to about 3,000 mg, about 2,950 mg to about 3,000 mg, about 2,960 mg to about 3,000 mg, about 2,970 mg to about 3,000 mg, about 2,980 mg to about 3,000 mg, or about 2,990 mg to about 3,000 mg.

In some embodiments, the cannabis extract comprises tetrahydrocannabinol (THC) and cannabidiol (CBD). In some embodiment, the THC and the CBD are present in the cannabis extract in a volume:volume ratio in a range of about 1:50 to about 1:1. In some embodiments, the ratio of THC to CBD can be, in a volume:volume ratio, at least about 1:1, at least about 2:1, at least about 3:1, at least about 4:1, at least about 5:1, at least about 6:1, at least about 7:1, at least about 8:1, at least about 9:1, at least about 10:1, at least about 11:1, at least about 12:1, at least about 13:1, at least about 14:1, at least about 15:1, at least about 16:1, at least about 17:1, at least about 18:1, at least about 19:1, at least about 20:1, at least about 21:1, at least about 22:1, at least about 23:1, at least about 24:1, at least about 25:1, at least about 26:1, at least about 27:1, at least about 28:1, at least about 29:1, at least about 30:1, at least about 31:1, at least about 32:1, at least about 33:1, at least about 34:1, at least about 35:1, at least about 36:1, at least about 37:1, at least about 38:1, at least about 39:1, at least about 40:1, at least about 41:1, at least about 42:1, at least about 43:1, at least about 44:1, at least about 45:1, at least about 46:1, at least about 47:1, at least about 48:1, at least about 49:1, or at least about 50:1. In some embodiments, the cannabis extract can comprise the active agent is cannabigerol (CBG), cannabichromene (CBC), cannabigerivarin (CBGV), tetrahydrocannabivarin (THCV), cannabidivarin (CBDV), and/or cannabichromevarin (CBCV).

In some embodiments, the H. erinaceus extract comprises one or more erinacines; one or more hericenones; or a combination thereof. In some embodiments, the one or more erinacines are erinancine A, erinancine B, erinancine C, erinancine D, erinancine E, erinancine F, erinancine G, erinancine H, erinancine I, erinancine J, erinancine K, erinancine P, erinancine Q, erinancine S, erinancine U, or a combination thereof. In some embodiments, the one or more hericenones are hericenone A, hericenone B, hericenone C, hericenone D, hericenone E, hericenone F, hericenone G, hericenone H, hericenone I, hericenone J, hericenone K, or a combination thereof.

The ranges and amounts above of cannabis extract and H. erinaceus extract are based on a volume in a range of from about 222 mL to about 355 mL of beverage composition total volume. Such ranges, however, contemplate that the total beverage volume can be any value, and thus the ranges and amounts of cannabis extract and H. erinaceus extract are modified accordingly. For example, in a 1 mL total volume of beverage extract, the range of cannabis extract can be from about 22.5 μg to about 45 μg, and the range of H. erinaceus extract can be from about 45 μg to about 13.51 mg.

Based on the range of from about 222 mL to about 355 mL of beverage compostion total volume, the beverage total volume can be about 222 mL, about 225 mL, about 230 mL, about 235 mL, about 240 mL, about 245 mL, about 250 mL, about 255 mL, about 260 mL, about 265 mL, about 270 mL, about 275 mL, about 280 mL, about 285 mL, about 290 mL, about 295 mL, about 300 mL, about 305 mL, about 310 mL, about 315 mL, about 320 mL, about 325 mL, about 330 mL, about 335 mL, about 340 mL, about 345 mL, about 350 mL, or about 355 mL. The beverage composition total volume can also be expressed as a range, e.g., of from about 222 mL to about 355 mL, about 225 mL to about 355 mL, about 230 mL to about 355 mL, about 235 mL to about 355 mL, about 240 mL to about 355 mL, about 245 mL to about 355 mL, about 250 mL to about 355 mL, about 255 mL to about 355 mL, about 260 mL to about 355 mL, about 265 mL to about 355 mL, about 270 mL to about 355 mL, about 275 mL to about 355 mL, about 280 mL to about 355 mL, about 285 mL to about 355 mL, about 290 mL to about 355 mL, about 295 mL to about 355 mL, about 300 mL to about 355 mL, about 305 mL to about 355 mL, about 310 mL to about 355 mL, about 315 mL to about 355 mL, about 320 mL to about 355 mL, about 325 mL to about 355 mL, about 330 mL to about 355 mL, about 335 mL to about 355 mL, about 340 mL to about 355 mL, about 345 mL to about 355 mL, about 350 mL to about 355 mL, about 222 mL to about 350 mL, about 222 mL to about 345 mL, about 222 mL to about 340 mL, about 222 mL to about 335 mL, about 222 mL to about 330 mL, about 222 mL to about 325 mL, about 222 mL to about 320 mL, about 222 mL to about 315 mL, about 222 mL to about 310 mL, about 222 mL to about 305 mL, about 222 mL to about 300 mL, about 222 mL to about 295 mL, about 222 mL to about 290 mL, about 222 mL to about 285 mL, about 222 mL to about 280 mL, about 222 mL to about 275 mL, about 222 mL to about 270 mL, about 222 mL to about 265 mL, about 222 mL to about 260 mL, about 222 mL to about 255 mL, about 222 mL to about 250 mL, about 222 mL to about 245 mL, about 222 mL to about 240 mL, about 222 mL to about 235 mL, about 222 mL to about 230 mL, or about 222 mL to about 225 mL.

In some embodiments, beverage composition can comprise one or more sour additives. In some embodiments, the sour additive is lactate, lactic acid, malate, malic acid, malonate, malonic acid, oxalate, oxalic acid, citrate, citric acid, acetate, acetic acid, fumarate, fumaric acid, phosphate, phosphoric acid, tartarate, tartaric acid, sorbate, sorbic acid, succinate, succinic acid, sodium acid sulfate, pyruvate, pyruvic acid, or a combination thereof.

In some embodiments, the beverage composition may further include sugar acids (e.g., glucuronic acid) and/or sugar alcohols (e.g., inositol). Sugar acids may be selected be from, e.g., glyceric acid, xylonic acid, gluconic acid, ascorbic acid, neuraminic acid, keto-deoxyoctulosonic acid, glucuronic acid, galacturonic acid, iduronic acid, tartaric acid, meso-galactaric acid, D-glucaric acid, or a combination thereof. Sugar alcohols may be selected from, e.g., mannitol, sorbitol, arabitol, threitol, xylitol, ribitol, galactitol, fruitol, iditol, inositol, volemitol, lactitol, malitol, or combinations thereof.

In some embodiments, amino acids may also be included in the beverage composition, e.g., L-phenylalanine, L-leucine, L-isoleucine, L-valine, L-arginine, L-histidine, L-aspartic acid, L-glutamic acid, L-serine, L-threonine, L-asparagine, L-glutamine, L-cysteine, L-selenocysteine, glycine, L-proline, L-alanine, L-methionine, L-tyrosine, and/or L-tryptophan.

In some embodiments, the beverage composition can include amino acid derivatives. Such amino acid derivatives can be, e.g., N-acetyl L-tyrosine, taurine, ornithine, sarcosine, citrulline, norvaline, norleucine, α-aminobutyric acid, hydroxyproline, tert-leucine, cycloleucine, α-aminoisobutyric acid (2-methylalanine), penicillamine, homoserine, or a combination thereof. Additionally, any of various derivatives of the amino acids and amino acid derivatives mentioned above can also be included in the compositions. Examples of these amino acid derivatives include, e.g., special amino acids, non-natural amino acids, amino alcohols, and amino acids one or more of which functional groups such as terminal carbonyl group, terminal amino group, and thiol group in the case of cysteine are substituted with any one or more of various substituents. Specific examples of the substituents include, for example, an alkyl group, an acyl group, hydroxyl group, amino group, an alkylamino group, nitro group, sulfonyl group, and various protective groups. Specific examples of these amino acid derivatives include, e.g., N-γ-nitroarginine, S-nitrocysteine, S-methylcysteine, S-allylcysteine, valinamide, 2-amino-3-methyl-1-butanol (valinol), methionine sulfoxide, and S-methylcysteine sulfoxide.

Some embodiments can comprise natural flavors and/or artificial flavors. Natural flavors can be an essential oil, oleoresin, essence or extractive, protein hydrolysate, distillate, or any product of roasting, fermenting, heating or enzymolysis, which contains the flavoring constituents derived from a spice, fruit or fruit juice, vegetable or vegetable juice, edible yeast, herb, bark, bud, root, leaf or similar plant material, meat, seafood, poultry, eggs, dairy products, or fermentation products thereof, whose significant function in food is flavoring rather than nutritional. Artificial flavors are any substance, the function of which is to impart flavor, which are not derived from a spice, fruit or fruit juice, vegetable or vegetable juice, edible yeast, herb, bark, bud, root, leaf or similar plant material, meat, fish, poultry, eggs, dairy products, or fermentation products thereof. Traditional artificial flavoring such as sodium citrate, ascorbic acid, diacetyl, acetylpropinyl, acetoin, isoamyl acetate, benzaldehyde, cinnamaldehyde, ethyl propionate, methyl anthranilate, limonene, ethyl decadienoate, allyl hexanoate, ethyl maltol, ethylvanillin, methyl salicylate, manazanate, or a combination thereof may be used.

Preservatives may also be included in the beverage composition. For example, in some embodiments, sodium citrate, potassium sorbate, sodium benzoate, potassium benzoate, ethylenediamine tetraacetic acid (EDTA), calcium disodium EDTA, sorbic acid, sodium metabisulfate, sodium sulfite, sodium nitrite, propyl paraben, butylated hydroxyanisole, butylated hydroxytoluene, citric acid, Vitamin E, Vitamin C, and/or benzoic acid may be included in the composition.

In some embodiments, beverage compositions may further include pH buffer(s), such as potassium phosphate, dipotassium phosphate, potassium hydrophosphate, sodium bicarbonate, sodium citrate, sodium phosphate, disodium phosphate, sodium hydrophosphate, sodium tripolyphosphate, potassium citrate, magnesium citrate, or a combination thereof.

Contemplated compositions may also include nutritional supplements including, but not limited to: magnesium carbonate, potassium phosphate dibasic, ascorbic acid, “super creatine”, coenzyme Q10 (CoQ10), citicoline, omega-3 fatty acids (such as, e.g., α-linolenic acid, eicosapentaenoic acid, docosahexaenoic acid, hexadecatrienoic acid, stearidonic acid, eicosatrienoic acid, eicosatetraenoic acid, heneicosapentaenoic acid, docosapentaenoic acid, tetracosapentaenoic acid, tetracosahexanenoic acid), omega-6 fatty acids (such as, e.g., linoleic acid, γ-linolenic acid, calendic acid, eicosadienoic acid, dihomo-γ-linolenic acid, arachidonic acid, docosadienoic acid, adrenic acid, osbond acid, tetracosatetraenoic acid, tetracosapentaenoic acid) lutein, zeaxanthin, L-theanine, fish oil, β-alanine, D-ribose, L-glutathione reduced, citrulline, L-alanyl-L-glutamine, L-hydroxyproline, or a combination thereof.

In some embodiments, the beverage composition includes herbal supplements, e.g., ginseng, milk thistle, guarana, gingko biloba, saw palmetto, green tea, black tea, hoodia gordonii, passion flower, horny goat weed, skullcap, Echinacea, dandelion leaf, St. John's wort, green tea, chamomile, or peppermint, or an extract thereof, or a combination thereof. In some embodiments a recovery supplement for hypoxic exercise, such as L-carnitine L-tartrate, creatine, whey protein, citrulline, curcumin, β-hydroxybutyrate, or a combination thereof is included in the composition. In some embodiments, a detoxifying agent, e.g., D-glucuronolactone, milk thistle, magnesium, diatomaceous earth, glutathione, alpha lipoic acid, superoxide dismutase, N-acetyl cysteine, selenium, or a combination thereof may be included in the beverage composition.

In some embodiments, the beverage composition includes electrolytes, e.g., sodium chloride, sodium acetate, acidic sodium citrate, acidic sodium phosphate, sodium chloride, sodium bicarbonate, sodium bromide, sodium citrate, sodium lactate, sodium molybdate, sodium phosphate, anhydrous sodium sulphate, sodium sulphate, sodium tartrate, sodium benzoate, sodium selenite, potassium chloride, potassium acetate, potassium bicarbonate, potassium bromide, potassium citrate, potassium-D-gluconate, monobasic potassium phosphate, potassium tartrate, potassium sorbate, potassium iodide, magnesium carbonate, magnesium citrate, magnesium oxide, magnesium phosphate, magnesium chloride, calcium chloride, calcium carbonate, calcium chelate, calcium di-phosphate, calcium lactate, calcium phosphate tribasic, or a combination thereof.

In some embodiments, the beverage composition includes colors (e.g., yellow #5, yellow #6, blue #1, blue #2, green #3, red #3, red #40, citrus red 2, orange B, caramel color, quinolone yellow (E104), carmoisine (E122), ponceau 4R (E124), patent blue V (E131), green S (E142), annatto extract, dehydrated beets, β-carotene, grape skin extract, or a combination thereof), gums (e.g., xanthan gum, gum Arabic, ester gum, guar gum, carob gum, gellen gum, cellulose gum, tara gum, or a combination thereof), and/or an emulsifier (e.g., monosodium phosphate, disodium phosphate, dipotassium phosphate, trisodium phosphate, sodium metaphosphate (sodium hexametaphosphate), sodium acid pyrophosphate, tetrasodium pyrophosphate, sodium aluminum phosphate, sodium citrate, potassium citrate, calcium citrate, sodium tartrate, sodium potassium tartrate, or a combination thereof).

In some embodiments, the beverage composition includes a bitter additive, e.g., branched amino acids such as L-leucine, L-Valine or L-isoleucine; quinine HCl and its salts; hesperidin; sucrose octaacetate; quercetin; brucine; quassin; isohumulones; stevia extracts; saccharin; naringin; gustducin; catechin; sesquiterpene lactone; aristolochic acid; phenylthiocarbamide; propylthiouracil; flavones; noscapine; humulone; amarogentin; glucoside; limonin; amarogentin; goitrin; cynaropicrin; quassin; cycloheximide; dipeptides; any other bitter-tasting compounds from vegetables, fruits (for example, naringin and guarana), cocoa (for example, chocolate), cereals, or spices; or a combination thereof.

In some embodiments, the beverage composition includes a stabilizer, e.g., agar, pectin, lecithin, egg yolk, soy lecithin, carrageenan, locust bean gum, gelatin, xanthan gum, alginin, guar gum, sodium pyrophosphate, or a combination thereof.

In some embodiments, the beverage composition is comprised in a beverage base. In some embodiments, the choice of beverage base may include, but is not limited to, water, tea, fruit juices, or other suitable liquids. The beverage base may include additional ingredients to ensure the stability, solubility, and/or palatability of the active components within the beverage base.

### Treatment Methods

In certain aspects, the disclosure relates to a method of reducing a withdrawal symptom and/or reducing an addictive behavior comprising administering a therapeutically effective amount of the beverage composition to a subject having the withdrawal symptom and/or the addictive behavior. In certain aspects, the disclosure relates to a method for treating alcoholism in a subject comprising administering a therapeutically effective amount of the beverage composition to the subject. In some embodiments, the beverage composition replaces alcohol consumption by the subject.

In certain embodiments, the withdrawal symptom and/or reducing an addictive behavior is reduced only with the beverage composition In certain embodiments, the withdrawal symptom and/or reducing an addictive behavior is reduced with the beverage composition and an additional agent. In certain embodiments, treatment with the additional agent is initiated at the same time as treatment with the beverage composition. In certain embodiments, the treatment with the additional agent is initiated after the treatment with the beverage composition is initiated. In certain embodiments, treatment with the additional agent is initiated at before the treatment with the beverage composition.

In some embodiments, the additional agent is methadone, buprenorphine, clonidine, naltrexone, or a combination thereof.

In certain embodiments, the beverage compositions of the present disclosure may be utilized for the treatment of withdrawal symptom and/or addictive behavior wherein the subject has failed at least one prior therapeutic regimen. Accordingly, the present disclosure provides methods of treating the withdrawal symptom and/or addictive behavior in a subject, wherein the subject has failed at least one prior therapeutic regimen for the withdrawal symptom and/or addictive behavior, comprising administering the beverage compositions as described herein to the subject in an amount sufficient to reduce the withdrawal symptom and/or addictive behavior, thereby treating the withdrawal symptom and/or addictive behavior. The beverage compositions described herein may also be utilized for inhibiting withdrawal symptoms and/or addictive behaviors in a subject wherein the subject has failed at least one prior therapeutic regimen.

For example, the beverage compositions described herein may be administered to a subject in an amount sufficient to reduce a withdrawal symptom and/or an addictive behavior relative to a subject that are not treated with the beverage composition. The beverage composition may reduce a withdrawal symptom and/or an addictive behavior by at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% relative to the withdrawal symptom and/or the addictive behavior that are not treated with the beverage composition.

In another aspect, the response from treatment with the beverage composition comprises a reduction in alcohol consumption. Examples of reduction in alcohol consumption include, but are not limited to reduction of heavy drinking, excessive drinking, drinks/day, percentage of subjects not drinking heavily, drinks/drinking day, percentage of subjects with no heavy drinking, and percentage of subjects who are abstinent. In another aspect, the method reduces the quantity of alcohol consumed compared with the amount of alcohol consumed before said treatment or compared with a control subject not receiving said treatment.

In another aspect, the method improves the physical or psychological sequelae associated with alcohol consumption compared with a control subject not receiving said treatment.

In another aspect, the method increases the abstinence rate of said subject compared with a control subject not receiving said treatment.

In another aspect, the method reduces the average level of alcohol consumption compared with the level before said treatment or compared with a control subject not receiving said treatment.

In another aspect, the method reduces alcohol consumption and increases abstinence compared with the alcohol consumption and abstinence before said treatment or compared with a control subject not receiving said treatment.

The concentration of withdrawal symptom and/or addictive behavior therapies can vary widely, and will be selected primarily based on fluid volumes, viscosities, body weight and the like in accordance with the particular mode of administration selected and the subject's needs. Generally, the amount of the withdrawal symptom and/or addictive behavior therapy or therapies present in a composition will be that which will produce a therapeutic effect. For example, in some embodiments, the weight per volume (w/v) or weight percent (wt %) concentration of a withdrawal symptom and/or addictive behavior therapy or therapies in the beverage composition may be between about 0.001% to 100%, 0.001% to 90%, 0.001% to 80%, 0.001% to 70%, 0.001% to 60%, 0.001% to 50%, 0.001% to 40%, 0.001% to 30%, 0.001% to 20%, 0.001% to 10%, 0.001% to 1%, 0.01% to 100%, 0.01% to 90%, 0.01% to 80%, 0.01% to 70%, 0.01% to 60%, 0.01% to 50%, 0.01% to 40%, 0.01% to 30%, 0.01% to 20%, 0.01% to 10%, 0.01% to 1%, 0.1% to 100%, 0.1% to 90%, 0.1% to 80%, 0.1% to 70%, 0.1% to 60%, 0.1% to 50%, 0.1% to 40%, 0.1% to 30%, 0.1% to 20%, 0.1% to 10%, 0.1% to 1%, 1% to 100%, 1% to 90%, 1% to 80%, 1% to 70%, 1% to 60%, 1% to 50%, 1% to 40%, 1% to 30%, 1% to 20%, 1% to 10%, 1% to 5%, 1% to 4%, 1% to 3%, 1% to 2%, 0.1% to 0.9%, 0.1% to 0.8%, 0.1% to 0.7%, 0.1% to 0.6%, 0.1% to 0.5%, 0.1% to 0.4%, 0.1% to 0.3%, 0.1% to 0.2%, 0.2% to 1%, 0.3% to 1%, 0.4% to 1%, 0.5% to 1%, 0.6% to 1%, 0.7% to 1%, 0.8% to 1%, or 0.9% to 1%.

One of ordinary skill in the art will appreciate that there are multiple parameters or characteristics of alcohol consumption which may characterize a subject afflicted with an alcohol-related disease or disorder. It will also be appreciated that combination therapies may be effective in treating more than one parameter, and that there are multiple ways to analyze the effectiveness of treatment. The parameters analyzed when measuring alcohol consumption or frequency of alcohol consumption include, but are not limited to, heavy drinking days, number of heavy drinking days, average drinking days, number of drinks per day, days of abstinence, number of individuals not drinking heavily or abstinent over a given time period, and craving. Both subjective and objective measures can be used to analyze the effectiveness of treatment. For example, a subject can self-report according to guidelines and procedures established for such reporting. The procedures can be performed at various times before, during, and after treatment. Additionally, assays are available for measuring alcohol consumption. These assays include breath alcohol meter readings, measuring serum carbohydrate-deficient transferrin (CDT) and gamma-glutamyl transferase (GGT) levels, and measuring 5-hydroxytryptophol (5-HTOL) urine levels.

A further aspect is for a method of producing altered perception and reduced stress while ameliorating a side effect of THC overconsumption in a subject comprising administering a therapeutically effective amount of the beverage composition to the subject.

Methods described herein may be used to treat a subject with a known or suspected side effect of THC overconsumption. Such side effects include, but are not limited to, hyperemesis syndrome, impaired coordination and performance, anxiety, suicidal ideations/tendencies, psychotic symptoms, dysphoria, panic, impairment of memory, reductions in psychomotor and cognitive performance, disordered perception of the passage of time, euphoria, schizophrenic psychosis, tiredness, dizziness, tachycardia, orthostatic hypotension, dry mouth, reduced lacrimation, muscle relaxation, and/or increased appetite. In some instances, this is determined by the judgement of a qualified healthcare professional or emergency medical technician. In some instances, a lack of or reduced response to prior treatments indicates a potential side effect of THC overconsumption.

In some embodiments, the subject shows signs of a side effect of THC overconsumption. In some embodiments, the side effect of THC overconsumption is caused by a compound other than THC from the Cannabis genus. In some embodiments, the side effect of THC overconsumption is caused by a synthetic cannabinoid. In some embodiments, the side effect of THC overconsumption is caused by oral ingestion of cannabinoids or synthetic cannabinoids. In some embodiments, the subject shows signs of cannabinoid hyperemesis syndrome.

In some embodiments, the method is capable of ameliorating one or more side effects of THC overconsumption in no more than 30 minutes. In another embodiment, the method is capable of ameliorating one or more side effects of THC overconsumption in no more than 1 hour. In various embodiments, the method is capable of ameliorating one or more side effects of THC overconsumption in no more than 10 minutes. In another embodiment, the method is capable of ameliorating one or more side effects of THC overconsumption in no more than 15 minutes. In various embodiments, the method is capable of ameliorating one or more side effects of THC overconsumption in no more than 45 minutes. In another embodiment, the method is capable of ameliorating one or more side effects of THC overconsumption in no more than 20 minutes. In various embodiments, the method is capable of ameliorating one or more side effects of THC overconsumption in 5-60, 5-45, 5-30, 5-25, 5-20, 5-15, 5-10, 10-60, 10-120, 10-30, 20-60, 20-120, 50-120, 60-120, or 90-120 minutes.

### EXAMPLES

### Example 1-Exemplary Formulation

Mother Liquor Emulsion is a full spectrum cannabis extract standardized to 2:1 CBD:THC ratio.

Lion's Mane-2,200 mg of fruiting bodies compressed, ultrasonically extracted, and nanoemulsified into a concentrated nanoemulsion.

A beverage compostion according to the present disclosure was prepared by mixing lion's mane extract, full-spectrum hemp extract, flavors, stabilizers, sweeteners, and water according to the proportions provided in Table 1.

TABLE 1

| Ingredient | Grade | Status | CAS | % |
|---|---|---|---|---|
| Lion's Mane | Food Grade | GRAS | N/A | 2.1% |
| Extract | | | | |
| Full-spectrum | NA | NA | 13956-29-1; | 2.0% |
| Hemp extract | | | 521-35-7; | |
| | | | 1972-08-3 | |
| Flavors | Food Grade | GRAS | | 29.3% |
| Stabilizers | Food Grade | GRAS | | 1.0% |
| Sweeteners | Food Grade | GRAS | | 34.1% |
| Water | Food Grade | GRAS | 144-55-8 | 36.1% |

### Example 2-Comprehensive Scientific Report: EEG Study on Theta Power Modulation by THC and Lion's Mane Extract

### BACKGROUND

Electroencephalography (EEG) measures electrical brain activity, with theta oscillations (4-8 Hz) linked to memory, attention, and emotional regulation (Buzsáki, 2002; Cha, 2024). Theta power is prominent in hippocampal and frontal regions, facilitating neural communication (Sauseng et al., 2008). Cannabinoids like Δ9-tetrahydrocannabinol (THC) modulate theta activity, often suppressing it (Ilan et al., 2004), while Hericium erinaceus (Lion's Mane) shows neuroprotective potential (Docherty et al., 2023). This study examines acute effects of 5 mg THC and 2.2 g Lion's Mane on theta power.

### Methods

Participants: 10 healthy adults (18-45 years) in a randomized, double-blind, crossover trial.

### Design:

Treatments: Baseline (placebo), Lion's Mane (2.2 g), THC (5 mg), THC+Lion's Mane.

Administration: Nanoemulsion beverages for rapid absorption.

EEG recordings: 16-node headset measured theta power at 0 min (baseline), 15 min, and 30 min.

### Analysis:

Normalized theta power analyzed via mixed-effects ANOVA.

Topographic mapping for spatial changes.

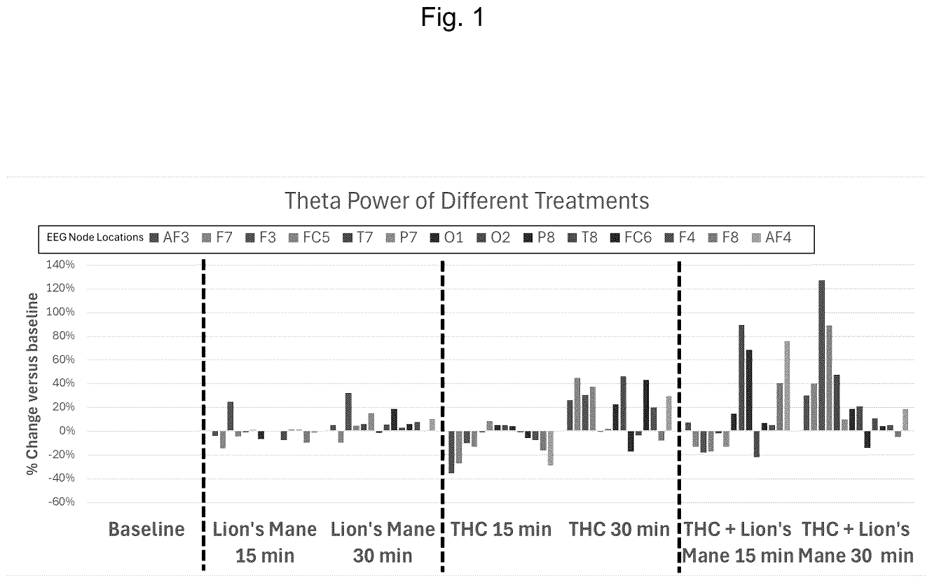

### Results (FIG. 1)

### Lion's Mane Alone:

Subtle, non-significant global theta increase (+7.2% at 30 min; p=0.18).

Frontal nodes (F3, F4) showed mild enhancement (+24.3%; p=0.11).

### THC (5 mg):

Significant global theta suppression at 15 minutes (−8.9% at 15 min; p<0.05), shifting to theta activation at 30 min (19.4%; p<0.05).

Parietal (P3, P4) and occipital (O1, O2) nodes increased 4-8% (p<0.05).

### THC+Lion's Mane:

Attenuated theta suppression (+15.8% global increase at 15 min and 28.7% at 30 min; p<0.05 vs. THC alone).

Frontal nodes (suppressed at 15 minutes F3, F7, and FC5: −10.8% vs. −14.6% for THC; p<0.05). Frontal nodes at 30 minutes significantly increased versus THC (65.2% vs. 33.2%

10/16 nodes differed significantly from THC (p<0.05).

### DISCUSSION

Low-Dose THC Effects: Even at 5 mg, THC significantly reduced theta power, aligning with dose-dependent CB1 receptor modulation (Wall et al., 2019). Posterior declines suggest disrupted parietal-occipital coherence (Bocker et al., 2010).

Lion's Mane Interaction: The 2.2 g dose enhanced mitigation of THC's effects, potentially via BDNF upregulation (Docherty et al., 2023) or anti-inflammatory pathways (Dimpfel et al., 2022). Frontal recovery highlights region-specific neuroprotection.

Limitations: Small sample size; single-dose design. Future studies should explore chronic dosing.

### CONCLUSION

5 mg THC suppressed theta power, while 2.2 g Lion's Mane attenuated this effect, particularly in frontal regions. Findings suggest Lion's Mane may buffer THC-induced neural changes, warranting further exploration for cognitive health applications.

### BIBLIOGRAPHY

Böcker KBE, Gerritsen J, Hunault C C, Kruidenier M, Mensinga T T, Kenemans J L. Cannabis with high Δ9-THC contents affects perception and visual selective attention acutely: an event-related potential study. Pharmacol Biochem Behav. (2010) 96:67-74. 10.1016/j.pbb.2010.04.008
Buzsáki, G. (2002). Theta oscillations in the hippocampus. Neuron. 2002 Jan. 31;33 (3): 325-40. doi: 10.1016/s0896-6273 (02) 00586-x. PMID: 11832222.
Dimpfel, W., Wiebe, J., & Gericke, N. (2022). Central Nervous System Profiling of Hericium erinaceus Biomass Powder by an Electropharmacogram Using Spectral Field Power in Conscious Freely Moving Rats. Clinical Journal of Mycology Vol VI Edition 1 . . . Ilan, A. B., Gevins, A., Coleman, M., ElSohly, M. A., & de Wit, H. (2004). Neurophysiological and subjective effects of smoked marijuana in frequent users. NeuroReport, 15 (17), 2729-2732
Docherty, S., Doughty, F. L., & Smith, E. F. (2023). The Acute and Chronic Effects of Lion's Mane Mushroom Supplementation on Cognitive Function, Stress and Mood in Young Adults: A Double-Blind, Parallel Groups, Pilot Study. Nutrients, 15 (22), 4842.
Böcker KB, Hunault C C, Gerritsen J, Kruidenier M, Mensinga T T, Kenemans J L. Cannabinoid modulations of resting state EEG θ power and working memory are correlated in humans. J Cogn Neurosci. 2010 September; 22 (9): 1906-16. doi: 10.1162/jocn.2009.21355. PMID: 19803687.
Wall M B, Pope R, Freeman T P, et al. Dissociable effects of cannabis with and without cannabidiol on the human brain's resting-state functional connectivity. Journal of Psychopharmacology. 2019; 33 (7): 822-830. doi: 10.1177/0269881119841568
Cha S, Bell L, Shukitt-Hale B, Williams C M. A review of the effects of mushrooms on mood and neurocognitive health across the lifespan. Neurosci Biobehav Rev. 2024 March; 158:105548. doi: 10.1016/j.neubiorev.2024.105548. Epub 2024 Jan. 19. PMID: 38246232.
Sauseng P, Klimesch W. What does phase information of oscillatory brain activity tell us about cognitive processes? Neurosci Biobehav Rev. 2008 July; 32 (5): 1001-13. doi: 10.1016/j.neubiorev.2008.03.014. Epub 2008 Apr. 18. PMID: 18499256.

### Example 3-Infused Beverage Study

The goal of the analysis was to answer a number of questions concerning participants experience with overall quality of life and alcohol consumption/desire before, during and after use of beverage compositions comprising 2.5 mg THC (approximately 10 mg cannabinoids total) and 2,200 mg of Lion's Mane, 355 mL total volume. Responses from participants were measured over time (22 days) comparing responses from the following data sets:
1. Enrollment
2. 7-days without the Lemon Elderflower Social Tonic (daily+weekly assessments)
3. 14-days with the Lemon Elderflower Social Tonic (daily+weekly assessments)
4. Final assessment on day 22.

The raw data at enrollment consisted of 95 participants, who answered a number of questions at different time points. Linear mixed-effects models that accounted for the dependent nature of the data (i.e., participants provided responses at multiple time periods, along with before and during product use, and these dependencies need to be accounted for) were utilized in all of the statistical analyses.

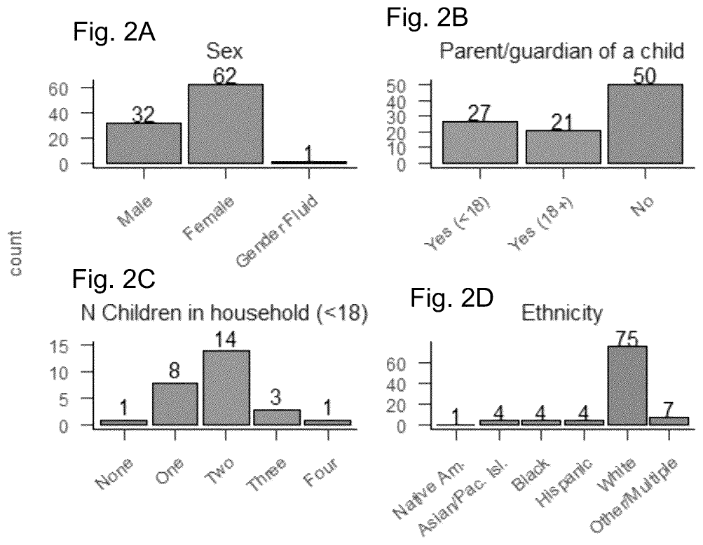

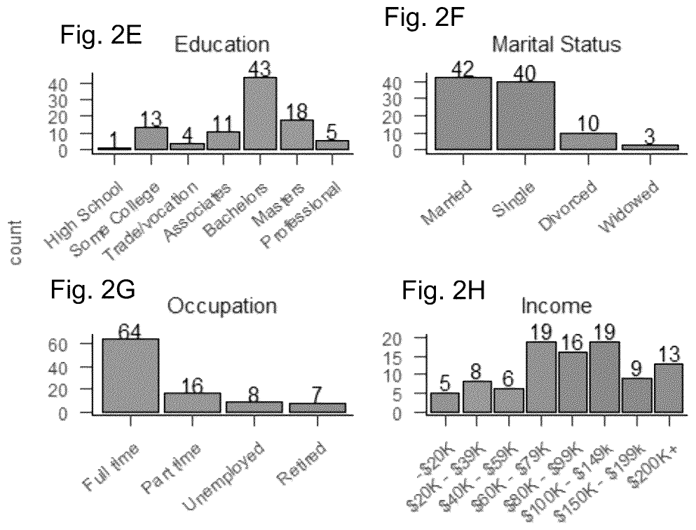

Below, Table 2 shows the number of participants who enrolled and completed the study and the demographic characteristics of the full sample (see FIG. 2A-H).

TABLE 2
Total number of participants who answered questions
at different stages of the campaign

| Category | N |
|---|---|
| Completed Enrollment | 108 |
| Weekly Assessment (Day 7) | 94 |
| Weekly Assessment (Day 14) | 92 |
| Weekly Assessment (Day 21 | 80 |
| Exit Survey | 76 |

### Weekly Scores

### Well-Being Weekly Score

Was there a change in Well-Being scores?
Answer: No, there was not a statistically significant change in Well-Being (p-value>0.05).

In the separate interaction models, dose, gender, and weight were not significant (p-value>0.05). This is a positive finding, as it suggests that the product is equally effective regardless of the dosage administered, the gender of the individual, or their body weight.

### Weekly Score for Sleep Disturbance

Was there a change in Sleep Disturbance scores?
Answer: Yes, there was a statistically significant change in Sleep Disturbance (p-value<0.05). This change was in the expected positive direction.

In the separate interaction models, dose, gender, and weight were not significant (p-value>0.05). This is a positive finding, as it suggests that the product is equally effective regardless of the dosage administered, the gender of the individual, or their body weight.

Sleep disturbance was measured using a validated research tool called the PROMIS Sleep Disturbance Short Form 8b. This questionnaire is widely recognized in the research community for assessing various aspects of sleep disturbance, including difficulty falling asleep, difficulty staying asleep, and sleep quality.

PROMIS sleep disturbance scores are standardized T-scores, where a score of 50 represents the average in the general population. Higher scores indicate greater sleep disturbance.

PROMIS threshold for minimally important difference is a 5 point difference. The average PROMIS score before using the product: 54.25

The average weekly score change: −0.85

The expected score for Sleep Disturbance upon completion: 51.7

This is a 2.55 point improvement in Sleep Disturbance (−4.69% change).

So, although there was statistically significant results (p-value<0.05), the threshold for a minimally important difference was not reached.

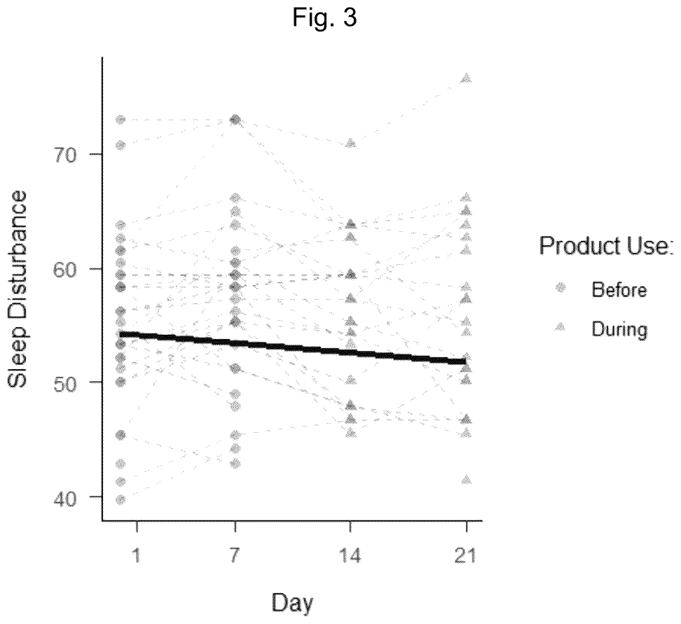

See FIG. 3 for a visual display of Sleep Disturbance scores per week.

### Weekly Score for DASS Depression

Was there a change in DASS Depression scores?
Answer: Yes, there was a statistically significant change in the DASS Depression (p-value<0.05). This change was in the expected positive direction.

In the separate interaction models, dose, gender, and weight were not significant (p-value>0.05). This is a positive finding, as it suggests that the product is equally effective regardless of the dosage administered, the gender of the individual, or their body weight.

Psychological distress was measured using the 21-item Depression Anxiety Stress Scales (DASS-21). This is a validated questionnaire widely used in the research community to assess symptoms of depression, anxiety, and stress (see Table 3).

Note that scores on each of the DASS-21 subscales (Depression, Anxiety, and Stress) range from 0=‘Did not apply to me at all’ to 3=‘Applied to me very much, or most of the time’. Higher scores indicate greater distress.

The average DASS Depression score before using the product: 8.93

The average weekly score change: −0.68

The expected score for upon completion: 6.88

This is a 2.05 point reduction in DASS Depression scores (−22.92% change).

TABLE 3

| Category | Depression | Anxiety | Stress |
|---|---|---|---|
| Normal | 0-9 | 0-7 | 0-14 |
| Mild | 10-13 | 8-9 | 15-18 |
| Moderate | 14-20 | 10-14 | 19-25 |
| Severe | 21-27 | 15-19 | 26-33 |
| Extremely Severe | 28+ | 20+ | 34+ |

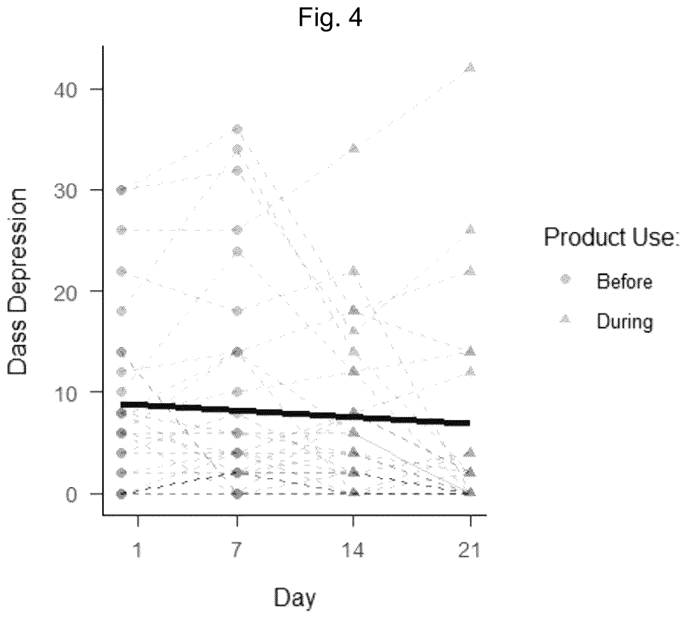

See FIG. 4 for a visual display of DASS Depression scores each week.

### Weekly Score for DASS Stress

Was there a change in DASS Stress scores?
Answer: Yes, there was a statistically significant change in the DASS Stress (p-value<0.05). This change was in the expected positive direction.

In the separate interaction models, dose, gender, and weight were not significant (p-value>0.05). This is a positive finding, as it suggests that the product is equally effective regardless of the dosage administered, the gender of the individual, or their body weight.

Psychological distress was measured using the 21-item Depression Anxiety Stress Scales (DASS-21). This is a validated questionnaire widely used in the research community to assess symptoms of depression, anxiety, and stress (see Table 3).

Note that scores on each of the DASS-21 subscales (Depression, Anxiety, and Stress) range from 0=‘Did not apply to me at all’ to 3=‘Applied to me very much, or most of the time’. Higher scores indicate greater distress.

The average DASS Stress score before using the product: 12.44

The average weekly score change: −1.13

The expected score for upon completion: 9.04

This is a 3.4 point reduction in DASS Stress scores (−27.36% change).

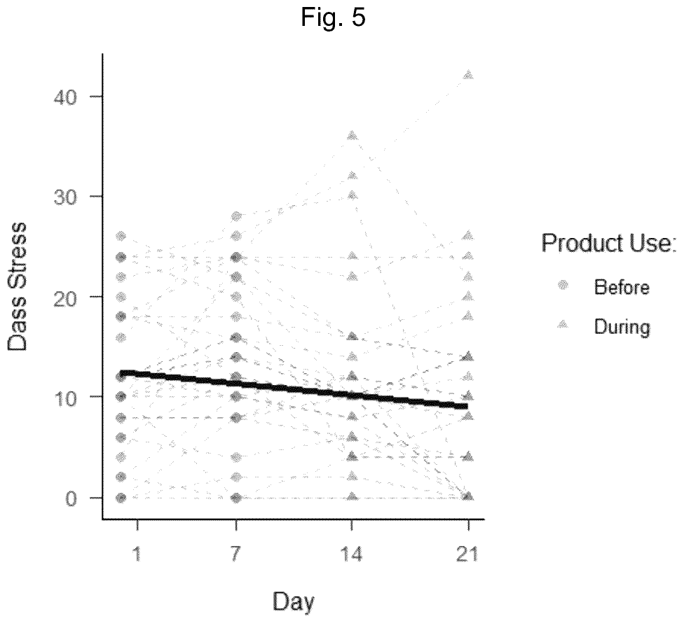

See FIG. 5 for a visual display of DASS Stress scores each week.

### Weekly Score for DASS Anxiety

Was there a change in DASS Anxiety scores?
Answer: Yes, there was a statistically significant change in the DASS Anxiety (p-value<0.05). This change was in the expected positive direction.

In the separate interaction models, dose, gender, and weight were not significant (p-value>0.05). This is a positive finding, as it suggests that the product is equally effective regardless of the dosage administered, the gender of the individual, or their body weight.

Psychological distress was measured using the 21-item Depression Anxiety Stress Scales (DASS-21). This is a validated questionnaire widely used in the research community to assess symptoms of depression, anxiety, and stress (see Table 3).

Note that scores on each of the DASS-21 subscales (Depression, Anxiety, and Stress) range from 0=‘Did not apply to me at all’ to 3=‘Applied to me very much, or most of the time’. Higher scores indicate greater distress.

The average DASS Anxiety score before using the product: 4.95

The average weekly score change: −0.67

The expected score for upon completion: 2.94

This is a 2.01 point reduction in DASS Anxiety scores (−40.56% change).

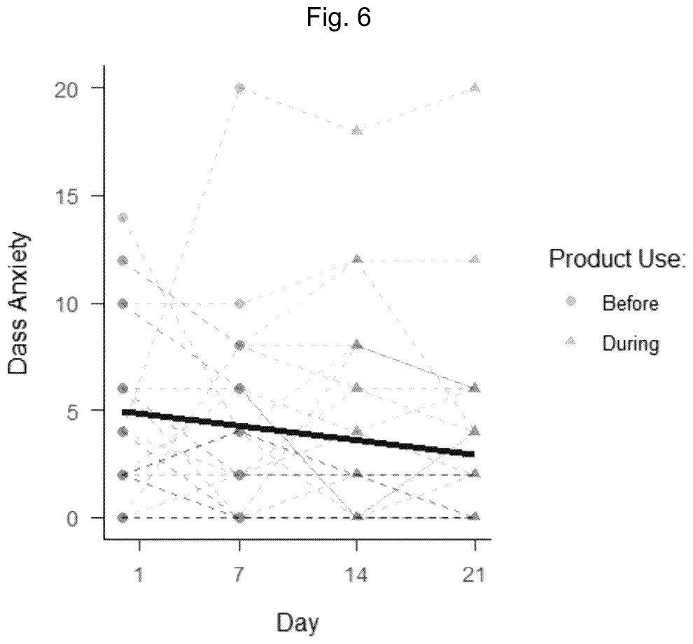

See FIG. 6 for a visual display of DASS Anxiety scores each week.

### Did You Consume Any Alcohol Yesterday? Daily Score

Was there a change in alcohol consumption?
Answer: Yes, there was a statistically significant change in the odds of alcohol consumption (p-value<0.05). This change was in the expected direction.

In the separate interaction models, dose, gender, and weight were not significant (p-value>0.05). This is a positive finding, as it suggests that the product is equally effective regardless of the dosage administered, the gender of the individual, or their body weight.

Note that scores on the measure are: Yes/No

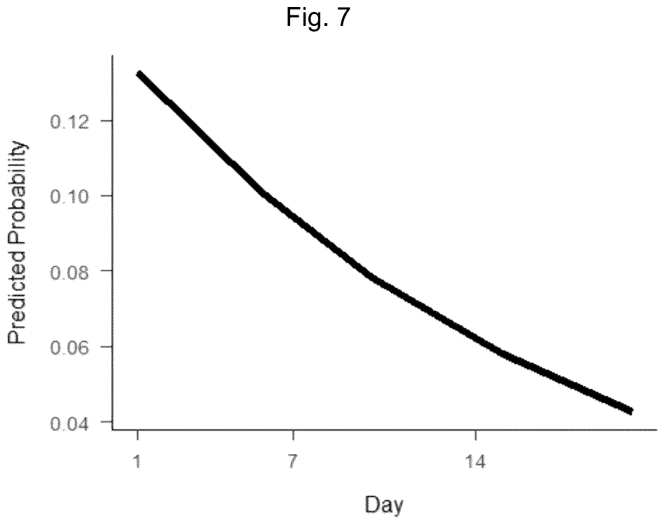

See FIG. 7 for a visual display of Alcohol Consumption predicted probabilities per week.

### How Many Drinks Did You Have Yesterday? Daily Score

Was there a change in number of alcohol drinks?
Answer: No, there was not a statistically significant change in Number Of Alcohol Drinks (p-value>0.05).

In the separate interaction models, dose, gender, and weight were not significant (p-value>0.05). This is a positive finding, as it suggests that the product is equally effective regardless of the dosage administered, the gender of the individual, or their body weight.

### How Many Hours of Sleep Did You Get Last Night?

Was there a difference in hours of sleep on days using the product vs days not using the product?
Answer: Yes, there was a statistically significant change in Sleep Hours (p-value<0.05). This change was in the expected positive direction.

In the separate interaction models, dose, gender, and weight were not significant (p-value>0.05). This is a positive finding, as it suggests that the product is equally effective regardless of the dosage administered, the gender of the individual, or their body weight.

The average hours of Sleep Hours without using the product: 6.9

The average hours of sleep when using the product: 7.13

This is a 0.23 point increase in sleep hours (3.21% change).

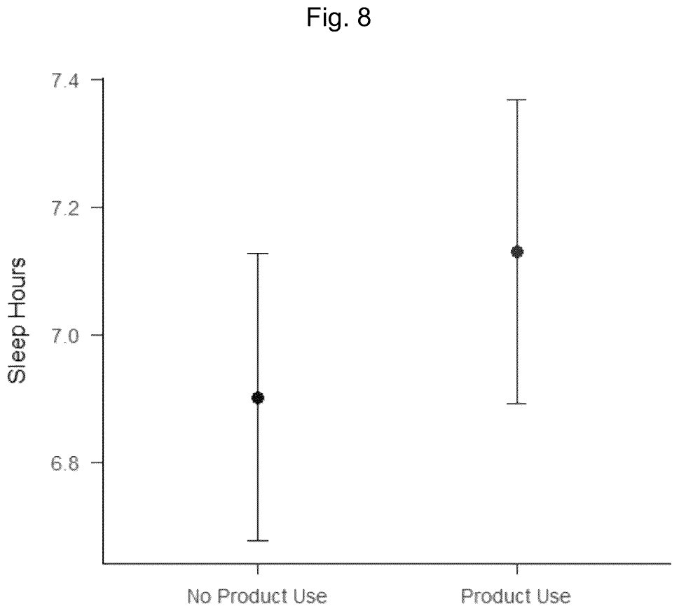

See FIG. 8 for a visual display of Sleep Hours scores per week.

What would You Rate Your Quality of Sleep Last Night?
Was there a difference in sleep quality on days using the product vs days not using the product?
Answer: Yes, there was a statistically significant change in Sleep Quality (p-value<0.05). This change was in the expected positive direction.

In the separate interaction models, dose, gender, and weight were not significant (p-value>0.05). This is a positive finding, as it suggests that the product is equally effective regardless of the dosage administered, the gender of the individual, or their body weight.

The average score for Sleep Quality without using the product: 5.55

The average score when using the product: 5.95

This is a 0.4 point increase in sleep quality (−6.66% change).

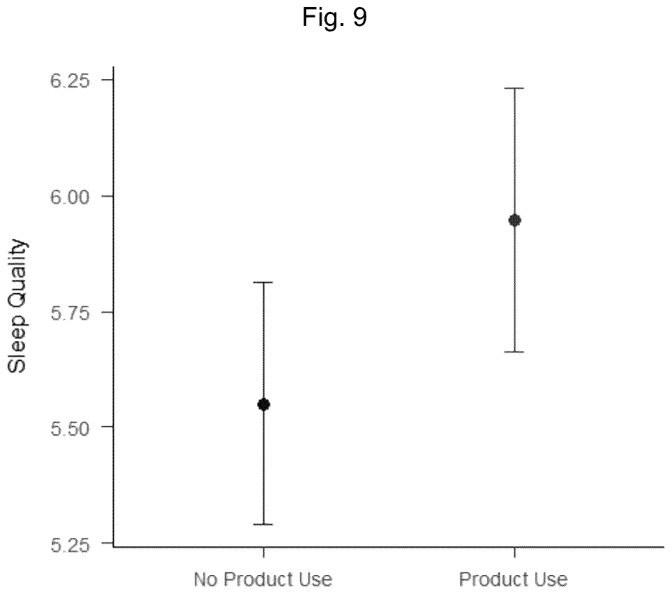

See FIG. 9 for a visual display of Sleep Quality scores per week.

How would You Rate Your Ability to Fall Asleep Last Night? was there a Difference In Ability to Fall Asleep on Days Using the Product
vs days not using the product?
Answer: Yes, there was a statistically significant change in Ability To Fall Asleep (p-value<0.05). This change was in the expected positive direction.

In the separate interaction models, dose, gender, and weight were not significant (p-value>0.05). This is a positive finding, as it suggests that the product is equally effective regardless of the dosage administered, the gender of the individual, or their body weight.

The average score for Ability To Fall Asleep without using the product: 2.91

The average score when using the product: 2.61

This is a 0.3 point increase in ability to fall asleep (11.5% change).

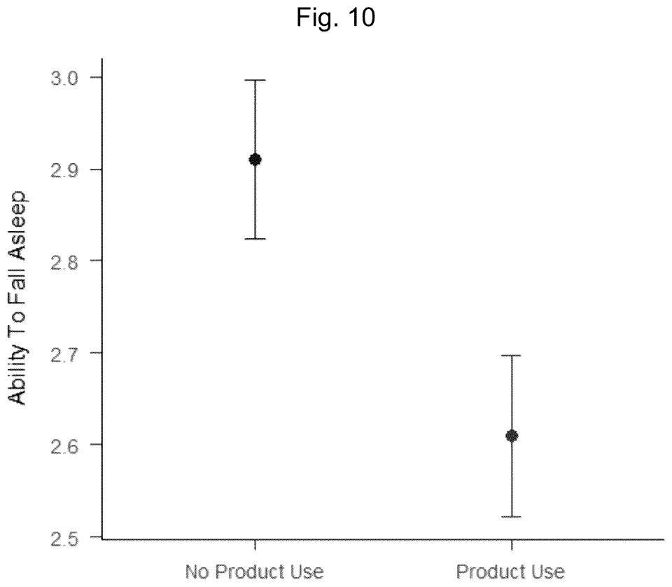

See FIG. 10 for a visual display of Ability To Fall Asleep scores per week.

What is claimed is:

1. A beverage composition comprising:
(a) from about 5 mg to about 100 mg of a cannabis extract; and
(b) from about 100 mg to about 3,000 mg of a Hericium erinaceus extract;
per about 222 mL to about 355 mL of beverage composition volume.

2. The beverage composition of claim 1, wherein the cannabis extract comprises tetrahydrocannabinol (THC) and cannabidiol (CBD).

3. The beverage composition of claim 2, wherein the THC and the CBD are present in the cannabis extract in a ratio in a range of about 1:50 to about 1:1.

4. The beverage composition of claim 1, wherein the H. erinaceus extract comprises:
(i) one or more erinacines;
(ii) one or more hericenones; or
(iii) a combination thereof.

5. The beverage composition of claim 4, wherein the one or more erinacines are erinancine A, erinancine B, erinancine C, erinancine D, erinancine E, erinancine F, erinancine G, erinancine H, erinancine I, erinancine J, erinancine K, erinancine P, erinancine Q, erinancine S, erinancine U, or a combination thereof.

6. The beverage composition of claim 5, wherein the one or more hericenones are hericenone A, hericenone B, hericenone C, hericenone D, hericenone E, hericenone F, hericenone G, hericenone H, hericenone I, hericenone J, hericenone K, or a combination thereof.

7. A method of reducing a withdrawal symptom and/or reducing an addictive behavior comprising administering a therapeutically effective amount of the beverage composition of claim 1 to a subject having the withdrawal symptom and/or the addictive behavior.

8. A method for treating alcoholism in a subject comprising administering a therapeutically effective amount of the beverage composition of claim 1 to the subject.

9. The method of claim 8, wherein the beverage composition replaces alcohol consumption by the subject.

10. A method of producing altered perception and/or reduced stress while ameliorating a side effect of THC overconsumption in a subject comprising administering a therapeutically effective amount of the beverage composition of claim 1 to the subject.
